(19) United States
(12) Patent Application Publication
(10) Pub. No.: US 2015/0184225 A1
(43) Pub. Date: Jul. 2, 2015

(54) ELECTROKINETIC POLYMERASE CHAIN REACTION (PCR) DEVICES AND METHODS

(71) Applicants: The Arizona Board of Regents on behalf of the University of Arizona, Tucson, AZ (US); Genefluidics, Inc., Irwindale, CA (US)

(72) Inventors: Pak Kin Wong, Tucson, AZ (US); Yi Lu, Tucson, AZ (US); Tingting Liu, Tucson, AZ (US); Vincent Gau, Irwindale, CA (US)

(73) Assignees: The Arizona Board of Regents on Behalf of the University of Arizona, Tucson, AZ (US); Genefluidics, Inc., Irwindale, CA (US)

(21) Appl. No.: 14/409,992

(22) PCT Filed: Jun. 25, 2013
(86) PCT No.: PCT/US13/47690

§ 371 Date: Dec. 19, 2014

Related U.S. Application Data
(60) Provisional application No. 61/690,382, filed on Jun. 25, 2012.

(51) Int. Cl. C12Q 1/68; B01L 7/00

(52) U.S. Cl. CPC C12Q 1/686; B01L 7/525; B01L 2200/10; B01L 2300/1833; B01L 2300/0645; B01L 2400/0415

(57) ABSTRACT

Described herein are microfluidic diagnostic methods and devices using electrokinetic modules for the isolation of targets (e.g., cells, bacteria, biomolecules) from biological samples, PCR amplification of DNA isolated from the targets, and real-time quantification of the amplified DNA using impedance sensing. Sample preparation, PCR amplification, and impedance sensing are thus performed using a single integrated platform.

### CROSS REFERENCE TO RELATED APPLICATIONS

This application claims the benefit of U.S. Provisional Application No. 61/690,382, filed Jun. 25, 2012, which is herein incorporated by reference in its entirety.

### ACKNOWLEDGMENT OF GOVERNMENT SUPPORT

This invention was made with government support under grant numbers OD007161, 1U01AI082457-01, and 2 R44AI088756-03 awarded by the National Institutes of Health. The government has certain rights in the invention.

### FIELD

This disclosure concerns microfluidic devices and methods that, inter alia, utilize electrokinetic forces to isolate and concentrate targets, such as biomolecules, cells or bacteria, and to carry out polymerase chain reaction (PCR) on target nucleic acid.

### BACKGROUND

Polymerase chain reaction (PCR) is a standard molecular biology technique used to amplify DNA starting from very small quantities of nucleic acid. PCR is widely used in a variety of research and clinical settings, such as to enable DNA sequencing, functional analyses of genes, diagnosis of genetic diseases, detection and diagnosis of infectious diseases, and DNA-based forensics.

Conventional PCR methods are carried out using a reaction mixture that includes template DNA, primers that hybridize to the template DNA, deoxynucleotide triphosphates (dNTPs) and DNA polymerase, all in a suitable buffer. The PCR method relies on thermal cycling, i.e., cycles of repeated heating and cooling to allow for DNA denaturation, annealing of primers to the DNA, and extension of the DNA template. The denaturation step typically occurs at a temperature of about 94-98° C., while the annealing step requires a temperature of about 50-65° C., and the extension step requires a temperature of about 72-80° C. This thermal cycling is generally repeated 20-40 times. As PCR progresses through each cycle, the DNA generated in the previous cycle(s) is itself used as a template for replication in the current cycle, thereby continuing a chain reaction in which the DNA template is exponentially amplified.

Detection of amplified PCR products can be accomplished using a number of different techniques. For example, PCR products can be detected by incorporating radiolabeled dNTPs and visualizing the products autoradiographically. Labeled (e.g., radiolabeled or fluorescently labeled) DNA probes that specifically hybridize to the amplified region of the DNA can also be used. Amplification of PCR products can also be confirmed by direct DNA sequencing. However, many techniques for detecting PCR products involve the use of expensive optics and contrast reagents. Another issue with conventional PCR is its typical requirement for the sample to be contained in a vial and physically transferred to different locations for performing respective temperature-critical steps. Yet another issue is the usual need to pre-process (e.g., purify or concentrate) a sample before commencing PCR, wherein concentration is typically performed in a different location and using different apparatus than used for PCR. Thus, a need exists for developing a rapid, cost-effective, and integrated system for sample pre-processing, PCR amplification, and quantification of amplified products.

### SUMMARY

The issues summarized above are resolved by various aspects and embodiments as described herein. For example, various embodiments provide simpler and faster PCR-sample preparation, as well as simpler and faster performance of PCR on the sample. PCR can be integrated with sample preparation, and any of various samples can be processed through PCR, including but not limited to biological fluids such as blood, serum, etc., and including samples containing biological cells. In addition, various embodiments allow monitoring of the progress of PCR, permitting PCR to be halted after it has been allowed to proceed to a particular concentration of amplified product.

Also disclosed herein are methods, apparatus, and systems that utilize electrokinetic forces for isolating target cells (such as bacterial cells), PCR amplification of nucleic acid obtained from the target cells, and impedance sensing for real-time detection and quantification of amplified DNA. The methods and systems disclosed herein possess several advantages compared to conventional PCR-based techniques, including the ability to concentrate pathogens of interest from complex biological samples and to amplify one or more specific genetic sequences from selected pathogens without sample pre-processing, shorter PCR cycles, and improved amplification efficiency. In addition, PCR products can be quantified in real time without the need for optics or contrast reagents.

Various embodiments include an enclosure for performing electrokinetic polymerase chain reaction (EK-PCR). The enclosure has walls defining an enclosed chamber in which an electrically conductive liquid can be contained that is formulated for performing PCR. A first set of electrodes contacts the conductive liquid in the chamber. The first set of electrodes, when electrically energized, can cause localized heating of the conductive liquid between the electrodes of the first set to produce an electrokinetic convective circulation of the liquid in the chamber. The convective circulation defines at least three temperature zones in the liquid in which denaturation, annealing, and extension phases, respectively, of PCR occur on a sample of genetic material suspended in the liquid. The convective circulation actually drives the sample to the different zones.

In other embodiments an electrokinetic chamber is provided, comprising a base, sides, and a cover defining an enclosed chamber in which an electrically conductive biological liquid can be contained, the chamber being dimensioned to support laminar flow of the biological liquid in the chamber. A set of electrodes is situated inside the chamber. The electrodes flank a cross-dimension (e.g. a midline) of the base to define first and second bilateral regions in the chamber. The electrodes when electrically energized can cause localized heating of liquid between the electrodes. An electrokinetic convection of the liquid in the chamber results by which first and second portions of the liquid circulate in the first and second regions, respectively, in a bilaterally symmetrical manner. Thus, the cells are concentrated in the vicinity of the electrodes.

In other aspects of the disclosure, a chamber for performing electrokinetic polymerase chain reaction (EK-PCR) is provided having a base, sides, and a cover defining an enclosed chamber for containing an electrically conductive liquid formulated for performing PCR. The chamber is dimensioned to support laminar flow of the liquid in the chamber. First and second electrodes are situated inside the chamber to define a circulation region in the chamber. When electrically energized, the first and second electrodes can cause localized heating of liquid between the electrodes. A resulting electrokinetic convection of the liquid is formed in the chamber by which at least a portion of the liquid circulates in the circulation region. The circulation defines at least three temperature zones in the region in which denaturation, annealing, and extension phases, respectively, of PCR can be performed on a sample of genetic material suspended in the liquid.

Since the area between the first and second electrodes is typically hottest in the chamber, the denaturation temperature zone usually extends at least between the electrodes.

In an embodiment, the denaturation temperature zone is about 94° C., the extension temperature zone is about 72° C., and the annealing temperature zone is about 55° C.

In another embodiment, a first set of electrodes flanks a midline of the base to define first and second bilateral regions in the chamber. The energized electrodes cause the liquid in the chamber to circulate in the first and second regions, respectively, in a bilaterally symmetrical manner to produce first and second, respectively, electrokinetic convective cycles of the liquid in the chamber.

In many instances the electrically conductive liquid comprises template DNA, primers, dNTPs, and DNA polymerase. The liquid may also include a PCR buffer.

The enclosure and/or chamber can further comprise a second set of electrodes including respective surfaces contacting the liquid inside the chamber. The respective surfaces of these electrodes comprises molecules of a DNA probe immobilized thereon.

In another embodiment, the enclosure and/or chamber comprises a second set of electrodes, wherein the first set of electrodes stimulates concentration of the sample and the second set of electrodes stimulates PCR on the genetic material of the sample.

The foregoing and other objects, features, and advantages of the invention will become more apparent from the following detailed description, which proceeds with reference to the accompanying figures.

### BRIEF DESCRIPTION OF THE DRAWINGS

FIG. 1A is a plan view of an embodiment of a sample concentrator that can also be used for performing electrokinetic (EK) PCR.

FIG. 1B is a plot showing the concentration of a sample of bacteria in the vicinity of the electrodes, versus time, performed using the embodiment of FIG. 1A.

FIG. 1C is a bright-field image of blood cells being urged away from the central electrodes of the concentrator shown in FIG. 1A.

FIG. 1D is a fluorescence image of fluorescently stained E. coli concentrated between the electrodes of the concentrator shown in FIG. 1A.

FIG. 2A is a plan view of an embodiment of a two-stage concentrator comprising interdigitated electrodes for increasing dielectrophoresis (DEP).

FIG. 2B shows motions of bacterial cells being subjected to DEP to produce a 100-fold volume reduction, using the embodiment of FIG. 2A. In a first step, bacteria are urged toward nearby pairs of electrodes by hybrid electrokinetics. In a second step, liquid in which bacterial cells are entrained flows to the right, transporting the bacteria to collection electrodes. In a third step, elution, the concentrated cells are passed out of the chamber. Liquid flow and bacteria trapping are produced by AC electrothermal flow (ACEF).

FIG. 3A is a perspective view of an embodiment of a EK-PCR chamber.

FIG. 3B schematically depicts thermal cycling achieved in the embodiment of FIG. 3A.

FIG. 3C is a plot of temperature versus distance to the right from the electrodes of the embodiment of FIG. 3A, as measured experimentally and calculated numerically.

FIG. 3D is a chromatic representation of the temperature distribution in the right side of the chamber of FIG. 3A, the representation being produced using dual-tracer fluorescence thermometry.

FIG. 3E schematically depicts an exemplary primer design for detecting E. coli 16S rRNA in a chamber such as the embodiment of FIG. 3A.

FIG. 3F is a photograph of a gel depicting DNA amplification by EK-PCR obtained using the chamber embodiment of FIG. 3A. Lane 1, 100-bp ladder; lane 2, negative control without template but with the Taq polymerase; lane 3, thermal-cycle product (35 cycles); lane 4, EK-PCR product in a 15 mm chamber; lane 5, EK-PCR product in a 12.5 mm chamber.

FIG. 4A depicts an exemplary velocity profile produced in the embodiment of FIG. 3A, illustrated by a plot of velocity versus distance from the gap between the electrodes. The curves may be used to estimate the time in which a unit of liquid containing PCR reagents is resident in each temperature zone established in the chamber.

FIG. 4B is a plot of an exemplary voltage dependence for controlling fluid velocity and cycle time in the embodiment of FIG. 3A.

FIG. 4C depicts an equivalent circuit model for predicting the complex impedance response of an in situ amplicon sensing electrode used in a chamber such as that shown in FIG. 3A.

FIG. 4D provides two plots depicting exemplary real-time monitoring of DNA hybridization using impedimetric sensing performed by amplicon-sensitive electrodes.

FIG. 5A depicts liquid flow in an alternative embodiment of a sample concentrator chamber exploiting EK-PCR. This alternative embodiment comprises three electrodes in parallel, wherein the middle electrode is electrically grounded and the flanking electrodes are energized by AC current. This embodiment can be used for both sample concentration and for performing EK-PCR.

FIG. 5B is a fluorescence image of fluorescently stained E. coli concentrated by the electrodes of the embodiment shown in FIG. 5A.

FIG. 5C is a bar graph showing the concentration of samples of three bacteria species in the vicinity of the electrodes using the embodiment of FIG. 5A, thereby illustrating the general applicability of hybrid electrokinetics for concentrating different bacteria including A. baumannii and B. globigii.

FIG. 5D is a bar graph of an exemplary increase in E. coli concentration in the vicinity of the electrodes of over 1000-fold in TAE (Tris-acetate-EDTA) buffer, over 1000-fold in urine, and over 300-fold in a buffy coat specimen using the embodiment of FIG. 5A.

FIG. 6 is a photograph of a gel depicting DNA amplification by EK-PCR obtained using the embodiment of FIG. 5A. Lane 1, 100-bp ladder; lane 2, thermal cycler produce (35 cycles); lane 3, negative control; lanes 4-8, EK-PCR product for durations of 15, 30, 60, 90 and 120 minutes, respectively.

FIG. 7A is a thermal diagram of a chamber similar to that of FIG. 5A. The thermal diagram is determined using infrared thermometry.

FIG. 7B provides plots of the temperature distribution as a function of distance for various chamber lengths in a chamber embodiment similar to that of FIG. 5A.

FIG. 8 schematically depicts the circulation of DNA template and PCR reagent through a temperature gradient established in a chamber embodiment such as that shown in FIG. 3A or FIG. 5A. FIG. 8 also depicts the incorporation of amplicon-sensitive impedance electrodes into the chamber with specific DNA capture probes immobilized thereto for real-time monitoring of DNA amplicon enrichment.

FIG. 9A provides a plot depicting impedance data of real-time EK-PCR. The time required for the impedance value to reach a desired threshold depends on the initial template concentration, thereby allowing quantification of the template DNA concentration.

FIG. 9B is a photograph of a gel electrophoresis showing correct amplification of the amplicon in the real-time EK-PCR system.

### DETAILED DESCRIPTION

As used in this application and in the claims, the singular forms “a,” “an,” and “the” include the plural forms unless the context clearly dictates otherwise. Additionally, the term “includes” means “comprises.” Further, the term “coupled” does not exclude the presence of intermediate elements between the coupled items.

The systems, apparatus, modules and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, modules, and apparatus are not limited to any specific aspect or feature or combinations thereof, nor do the disclosed systems, methods, modules, and apparatus require that any one or more specific advantages be present or problems be solved. Any theories of operation are to facilitate explanation, but the disclosed systems, methods, modules and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, modules and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art. In some examples, operations are discussed as occurring in a common workplace, but any combination of operations and any individual operations can be performed remotely from other operations and combinations thereof via one more wired or wireless networks or using point-to-point communication.

### I. ABBREVIATIONS

AC alternating current

ACEF alternating current electrothermal flow

EK electrokinetic

EP electrophoresis

DEP dielectrophoresis

PCR polymerase chain reaction

qPCR quantitative PCR (or real-time PCR)

rRNA ribosomal RNA

RT reverse transcriptase

### II. TERMS AND METHODS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Dielectrophoresis (DEP): A phenomenon in which a force is extended on a dielectric particle when it is subject to a non-uniform electric field. The force does not require the particle to be charged; all particles exhibit DEP activity in the presence of electric fields, but the exhibited DEP activity depends upon characteristics of the particles (size and shape), of the field (voltage and frequency), and other variables (composition and surfaces of the particles).

Electrokinetic: Refers to the motion of particles or fluids that are subjected to a difference in electrical potential, or the use of electrical current to create such movement, such as movement of particles or fluids.

Electrical potential: The work per unit charge necessary to move a charged body in an electrical field from a first point to a second point, measured in volts. The electrical potential (V) is related to current (I) and resistance (R) by the relationship V=IR. Herein, one or more pairs of electrodes in a chamber are energized by an AC electrical potential imposed across the electrodes.

Electrophoresis (EP): The movement and/or separation of suspended molecules and/or particles in a fluid as driven by an electrical potential applied by electrodes across the fluid. Molecules and particles are separated from each other according to their size and electrical charge.

Impedance sensing: A detection/quantification method based on measuring, using at least one pair of electrodes contacting the liquid in a PCR chamber, opposition to the flow of an alternating current applied across the electrodes.

Polymerase chain reaction (PCR): A method that allows for the amplification of nucleic acid starting from a single or very few copies of DNA. PCR relies of thermal cycling—cycles of repeated heating and cooling to allow for DNA denaturation, annealing of primer to the DNA, and extension of the DNA template. The denaturation step typically occurs at a temperature of about 94-98° C., which the annealing step requires a temperature of about 50-65° C. and the extension step requires a temperature of about 72-80° C. If Taq polymerase is used, extension is typically performed at about 72° C. Primers containing sequences complementary to the target region, along with a DNA polymerase, are key components to enable selective and repeated amplification of a target DNA. Typically, PCR employs a heat-stable DNA polymerase, such as Taq polymerase. As PCR progresses, the DNA generated is itself used as a template for replication, setting in motion a chain reaction in which the DNA template is exponentially amplified.

### III. DESCRIPTION OF REPRESENTATIVE EMBODIMENTS

The following descriptions of several representative embodiments are provided to illustrate certain particular features of the embodiments. These descriptions should not be construed to limit the disclosure to the particular features described.

Certain embodiments described below relate to electrokinetic (EK) modules, including hybrid EK modules, useful for microfluidic diagnostics. Hybrid EK combines long-range EK-induced fluid motion in a chamber with short-range EK-band trapping forces to allow effective manipulation of physiological samples. Certain embodiments exploit EK forces to enhance one or more of the following: (1) effective manipulations of the liquid in a chamber achieving large-scale volume reduction and isolation of a sample of interest; (2) isolating pathogens from complex matrices (e.g., urine and blood samples) without having to perform separate sample pre-processing; (3) enhancing PCR amplification efficiency by utilizing convectional flow generated in a chamber by electrohydrodynamics; and (4) in situ EK enhancement achieved using electrochemical sensors.

### First Representative Embodiment

A first embodiment comprises a chamber for performing isolation and concentration of specific targets (e.g., mammalian cells, bacteria, or biomolecules) from high-conductivity biological samples, such as urine or blood. For example, this embodiment concentrates a sample of bacteria by volume reduction (e.g., 10-fold to approximately 0.125 mL), thereby facilitating fluid handling and downstream PCR processing. Sample concentration is performed by exploiting hybrid electrokinetics (abbreviated hybrid EK).

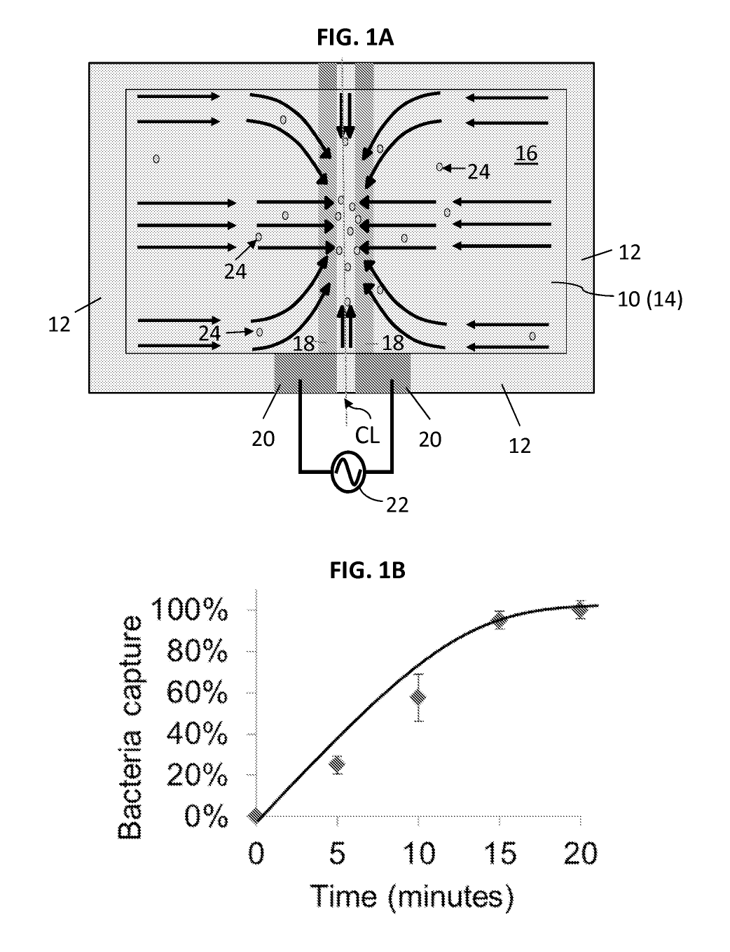

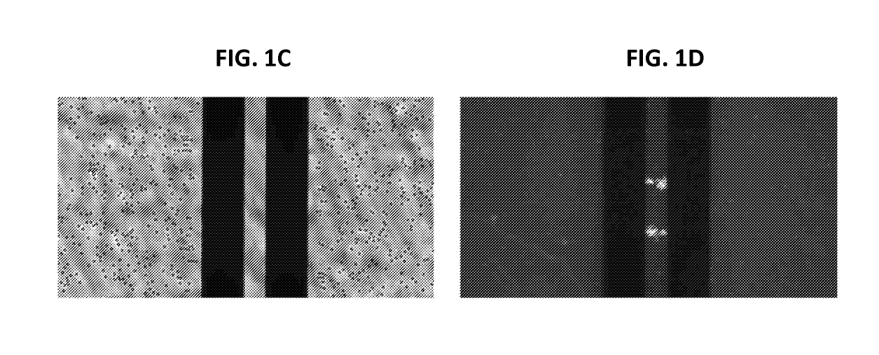

FIG. 1A is a schematic diagram of this embodiment of a hybrid EK concentrator. This embodiment comprises a base 10, sides 12, and a top 14 that collectively define a chamber 16. The chamber 16 has a mid-line CL representing a cross-dimension of the chamber 16. Flanking the mid-line CL are electrodes 18 extending across the base 10. The electrodes 18 have external pads 20 for connection to an external AC power supply 22. Appropriate energization of the electrodes 18 produces, by hybrid EK, a force profile (arrows) in the chamber that urges movement of cells 24 to the electrodes 18, particularly to the middle regions of the electrodes. As a result, the volume of liquid at the middle region of the electrodes 18 is enriched in cell concentration. In this embodiment, cells can be concentrated at the electrodes 18 using a combination of electrophoresis (EP), dielectrophoresis (DEP), and AC electrothermal flow (ACEF). During motion under hybrid EK conditions, fluid motion generated by ACEF entrains and urges motion of cells in a sample to the area near the electrode surfaces, where DEP is most effective. Movement forces extend to the walls 12 of the chamber. The resulting long-range fluid flow allows DEP to be effective in concentrating physiological samples (>1 S/m), which enhances the effective range of target-cell manipulation.

As seen in FIG. 1A, the hybrid EK concentrator of this embodiment comprises a chamber comprising a set of electrodes 18. Desirably, the chamber is bilateral, with the electrodes 18 being located approximately at chamber mid-length and flanking the cross-dimension of the chamber. The chamber may thus be bilaterally symmetrical. Flow of liquid established within the chamber desirably is laminar flow. One or more ports may be placed on the top 10 or bottom 14 for use in sample loading or removal.

This chamber may also be used for EK-PCR, in which a substantially circular (e.g., oval-shaped) flow is established between each electrode 18 and its corresponding side wall 12 (i.e., between the right electrode and the right wall and between the left electrode and the left wall). In an alternative embodiment (not shown), the chamber is substantially unilateral with the electrodes being located at one end of the chamber.

The hybrid EK-generated force profile may be tailored for handling a variety of sample volumes (such as, but not limited to, about 1.25 mL, or about 1.0 mL) and reducing PCR processing time to less than about 12 minutes, less than about 11 minutes, less than about 10 minutes, less than about 9 minutes, or less than about 8 minutes. For sample concentration, the chamber may have any of various heights, so long as the chamber can support substantially laminar flow. In various embodiments, the height may be chosen based on the effective range of ACEF fluid motion.

FIGS. 1B-1F provide results obtained using an EK-concentrator embodiment comprising two electrodes. See Example 1 for full descriptions of each Figure and the results contained.

### Second Representative Embodiment

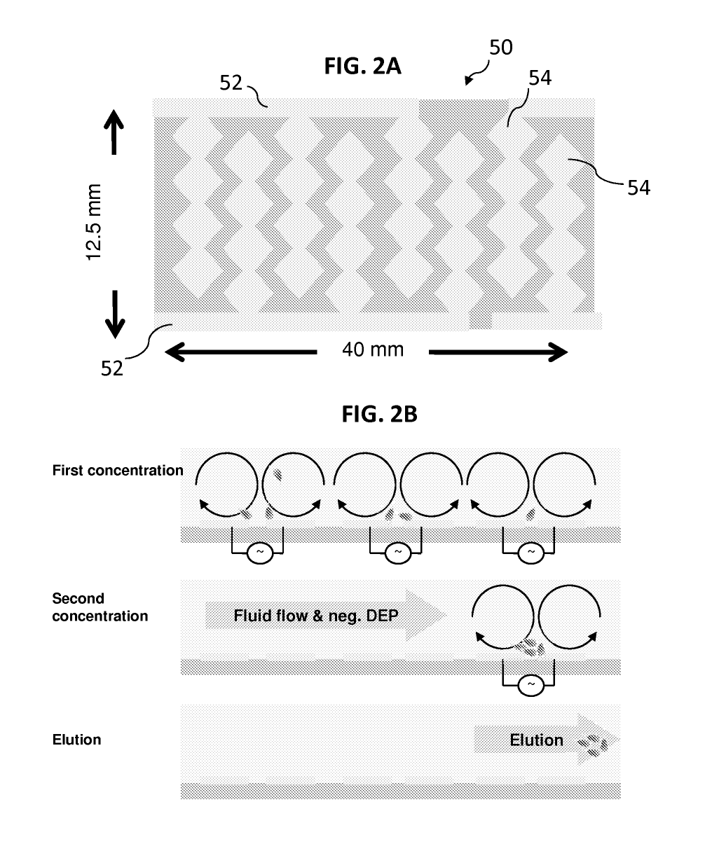

This embodiment is depicted in FIG. 2A and comprises a chamber 50 comprising first interdigitated electrodes 52 and second interdigitated electrodes 54. To handle 1.25 mL of the sample, the width and length of the chamber are 12.5 and 40 mm, respectively. As shown in FIG. 2A, the interdigitated electrodes are effective for concentrating a suspension of bacteria in the chamber (see below). The electrode array can significantly increase throughput, compared to a chamber comprising a pair of simple, non-digitated electrodes. A fluid velocity of ˜100 μm/sec may be attained in regions remote from the electrode, with over 99% of bacteria captured. With a chamber height (from the plane of the page) of 2 mm, substantially all pathogens are transported to regions near the electrode surfaces within about 20 seconds. To maximize capture efficiency, a safety factor may be applied. In one embodiment, the safety factor is approximately three, wherein the first concentration step is finished within about one minute.

As shown in FIG. 2B, multi-step concentrations may be performed using an electrode array such as in FIG. 2A. Specifically, multiple concentration steps may be applied to further concentrate the bacteria. The repetitive steps are established by energizing the first pair of electrodes 52, then energizing the second pair 54. Fluid flow may be produced from an external manifold system connected to the chamber 50. An AC potential applied to the interdigitated electrodes 52, 54 creates DEP forces useful for collecting the bacteria. Due to the initial position of the bacteria (near the electrodes) and the laminar nature of the flow, the bacteria can be effectively captured and concentrated in the chamber. DEP forces are strongest near the edges of electrodes. The flow velocity near the surface may be increased, such as to 200 μm/sec (equivalent to ˜4 pN hydrodynamic drag force), to ensure that all or substantially all bacteria are captured by the outer electrode. If necessary, negative DEP can be applied in the second inter-digitated electrode pair 54 to accelerate the second concentration step. After bacteria are captured by the second electrode pair 54, the sample can be eluted out, after removing the AC potential with a small volume (e.g. 125 μl) of liquid. Thus, for example, a 10-fold volume reduction of sample volume and corresponding sample environment is performed in a total processing time of less than 10 minutes.

The target may be pre-stained with a viability indicator (e.g., DiOC₆ (Sakakibara and Adrian, Experiments in Fluids 27(1):U1, 1999)) to facilitate identification and enumeration of cells captured by hybrid EK. To maximize the ACEF effect, the geometry of the interdigitated electrode array may be optimized, including the electrode surface area, shape and gap distance. A large surface area of electrodes maximizes the temperature gradient to enhance ACEF, while a small gap distance will increase the electric field strength (E=V/d). The interdigitated electrodes may have sharp corners which may increase DEP effects for bacteria trapping. A variety of electrode shapes, such as diamond, zig-zag, and cross-shaped, may be used for the interdigitated electrodes. Operating parameters may be optimized, such as the voltage, frequency, DC offset, and buffer conductivity for adjusting the relative strengths of DEP, EP, and ACEF. For example, a frequency dependence for capturing E. coli has previously been determined, usually approximately 1 MHz. The relative strength of DEP and ACEF may be fine-tuned by adjusting the applied voltage and frequency. Furthermore, the DC offset may create a time-averaged non-zero EP force for further enhancing the trapping effect.

This embodiment of an EK volume-reduction (concentration) enclosure allows, for example, a 10-fold volume reduction within 10 minutes (1 minute for the first concentration step; 6.7 minutes for the second concentration step, with elution occurring in less than 1 minute). Based on our studies, over 95% efficiency of sample capture can be achieved. In various embodiments, over 85%, over 90%, over 93%, over 96%, over 97%, or over 99% capturing efficiency is achieved.

### Third Representative Embodiment

This embodiment is directed to an EK-PCR chamber for amplifying specific nucleic acid sequences, such as bacterial 16S rRNA and corresponding DNA for example. The EK-PCR capability can be integrated with an EK-based sample-concentration capability in the same chamber. The EK-PCR chamber of the embodiment combines EK-induced fluid motion and a controlled temperature gradient created by Joule heating). EK-PCR has several advantages over other PCR techniques, including without limitation: (i) EK-induced fluid motion circulates liquid in the chamber to different temperature zones to eliminate thermal cycling and reducing PCR cycle time; and (ii) the temperature gradient is controlled for performing PCR reactions to avoid temperature non-uniformity typically observed in conventional microPCR. Furthermore, EK-PCR can be performed using very small electrodes, which allows effective integration with concentration-functions cost-effectiveness, (in contrast to expensive optics), and rapidity of detection (<1 hour) (e.g., of bacterial pathogens in various medical settings, and other resource-limited settings).

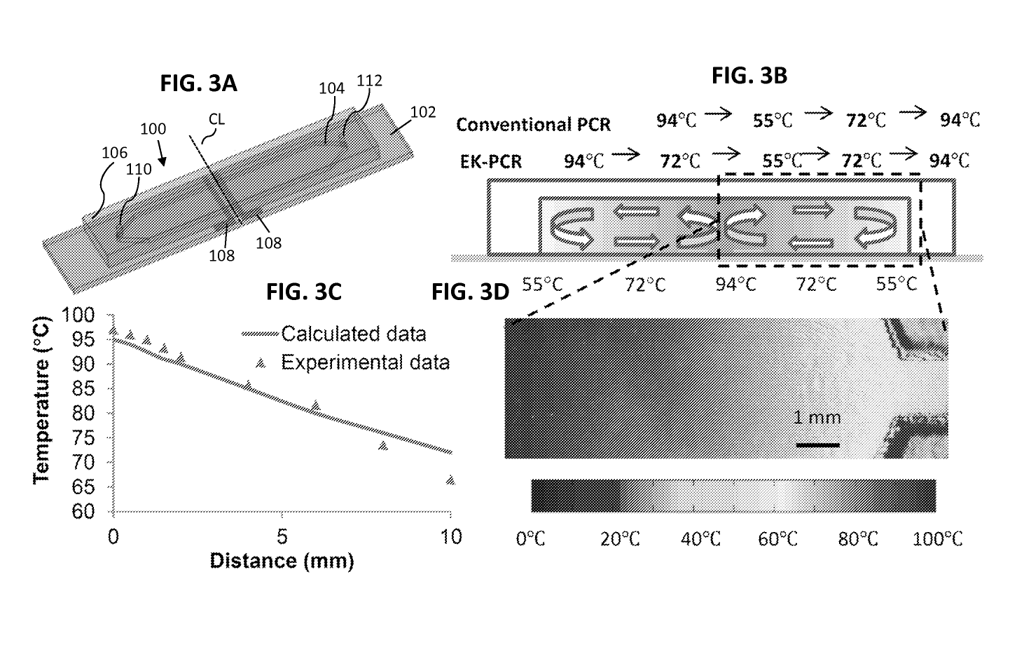

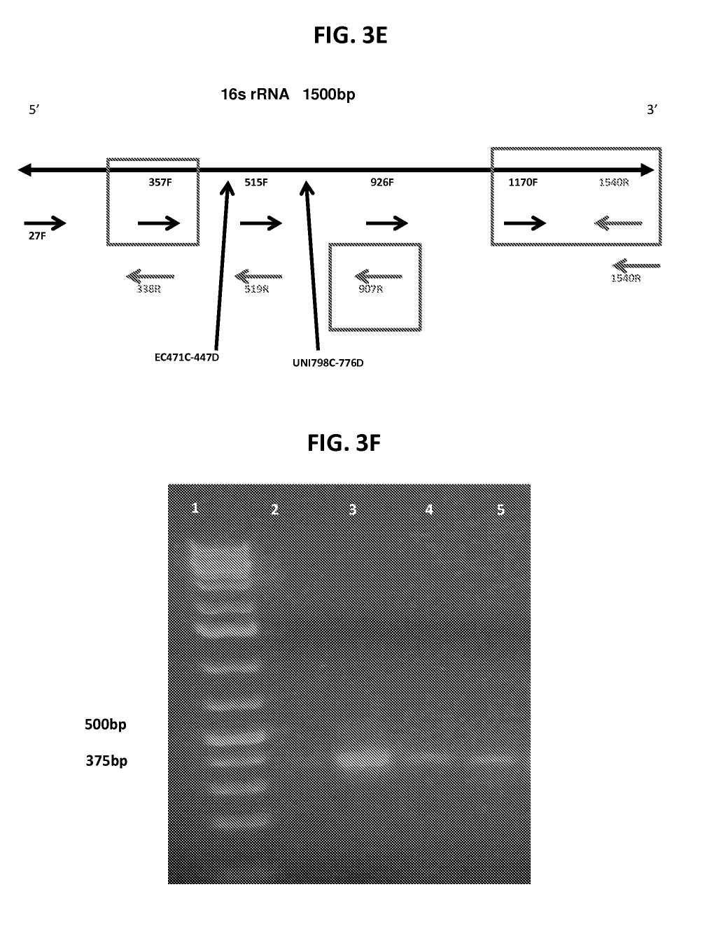

An EK-PCR chamber according to this embodiment is shown in FIG. 3A. The chamber 100 comprises a base 102 and combined sides 104 and top 106. A pair of chambers 108 extends across the base 102 to contact liquid in the chamber. In this embodiment, the electrodes 108 flank a cross-dimension CL of the chamber at substantially mid-length of the chamber. Ports 110 and 112 are defined in the top 106 to allow introduction of liquid to and the removal of liquid from the chamber. A port (not shown) can be located in the vicinity of the electrodes 108 for sample introduction. During use for PCR, the chamber is filled with an electrically conductive liquid (e.g., PCR buffer). During use, an AC potential is applied by the electrodes 108 to the liquid. A temperature rise is induced near the electrodes due to Joule heating of the liquid in the chamber, and a stable temperature gradient is formed as a result of heat diffusion and heat loss to the walls of the chamber (FIG. 3B). To estimate the temperature profile inside the chamber, a heat-transfer equation with heat loss to the top and bottom substrates can be considered:

$$kH \frac{{\partial }^{2} T}{{\partial }^{2} x} - {h}_{total} ( {T}_{0} - T ) + σ {E}^{2} = 0 ,$$

where hₜₒₜₐₗ is the heat-transfer coefficient of the chamber, k is the thermal conductivity of the liquid, and H is the height of the chamber.

In various embodiments, to perform PCR, the denaturation temperature zone may have temperatures between 94° C. and 98° C.; the annealing temperature zone may have temperatures between 50° C. and 65° C.; and the extension temperature zone may have temperatures between 72° C. and 80° C. The temperature distribution may be substantially continuous or, alternatively, may have one or more areas defining a substantially discontinuous or abrupt transition in temperature.

In one embodiment, to perform PCR, a continuous temperature distribution from 94° C. (denaturation) to ˜55° C. (annealing) to 72° C. (extension) is created in the chamber by applying to the electrodes a potential of 7 V at 1 MHz, as characterized by numerical calculation and dual-tracer fluorescence thermometry (FIGS. 3C-3D) (Kim and Yoda, Experiments in Fluids 49:257-266, 2010). An EK-induced fluid motion is produced by a hot core being produced just above and in the vicinity of the electrodes. The hot core has the highest temperature of any region in the chamber. As the hot liquid rises and encounters the top 106, it flows outward and is simultaneously cooled, thereby forming multiple temperature zones in the chamber. The hottest temperature core (approx. 94° C.) is located at the electrodes, which (for PCR) serves as a denaturation region. An intermediate temperature zone (approx. 72° C.) is situated laterally outward from the electrodes, which (for PCR) serves an an extension region. A third temperature zone (approx., 55° C.) is situated at the farthest lateral distance from the electrodes, which (for PCR) serves an an annealing region. The fluid in the chamber circulates between the denaturation region, the annealing region, and through the extension region, and executes PCR thermal cycles automatically (FIG. 3B). Using the embodiment, primers for detecting species-specific regions of bacterial 16S rRNA have been constructed, and EK-PCR for detecting E. coli using the primers has been demonstrated (FIG. 3E). FIG. 3F shows results obtained from two non-optimized EK-PCR chambers with 2 hours processing time, which correctly amplified product to a detectable concentration.

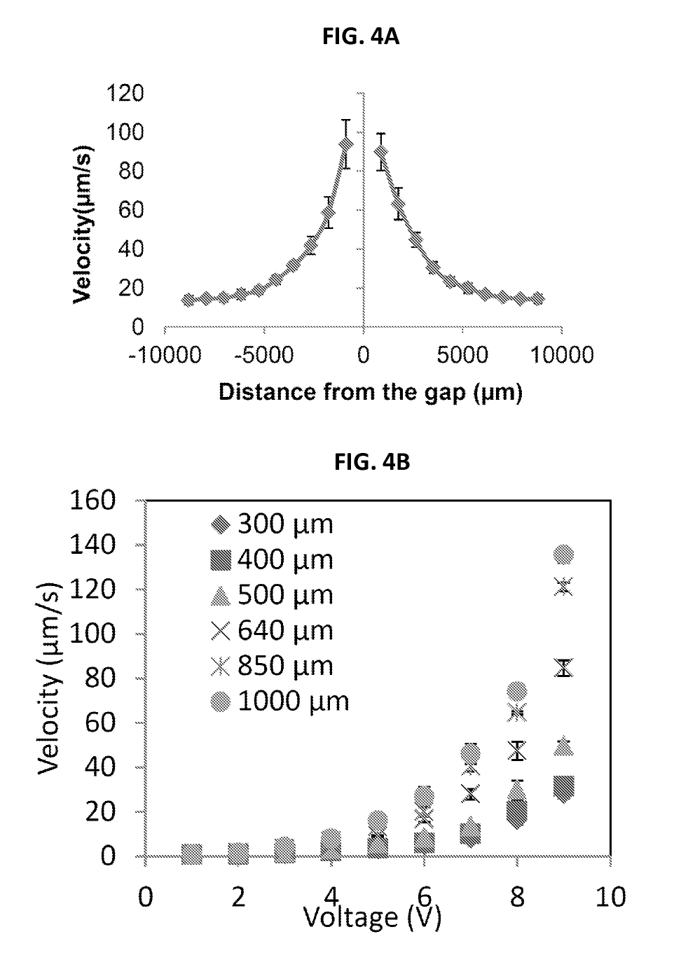

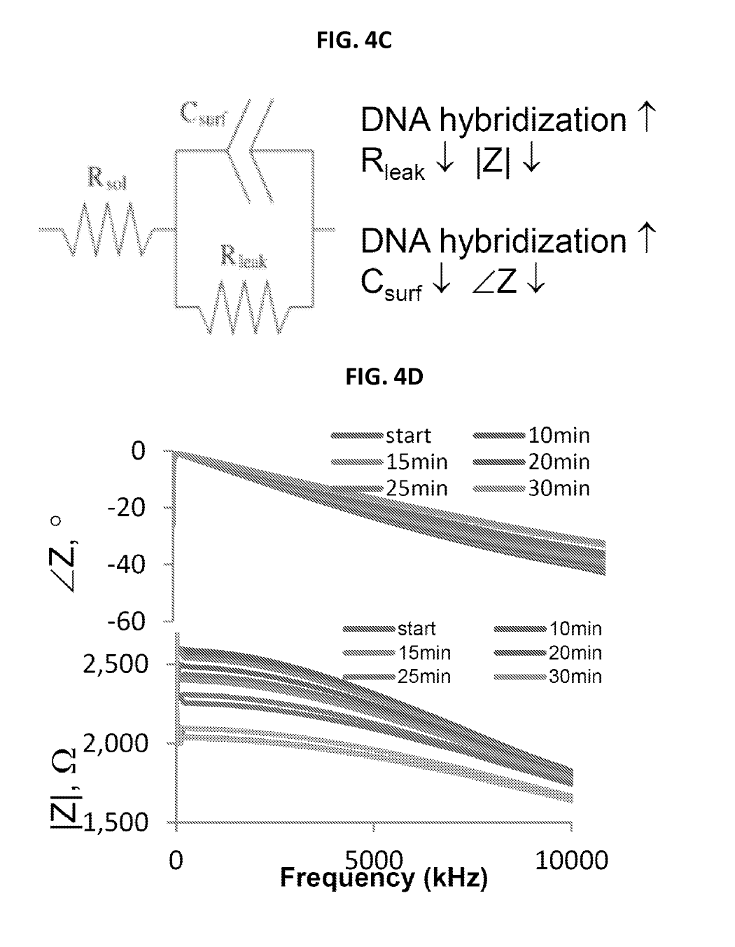

The EK-PCR chamber and the electrode geometry may be optimized by controlling the temperature and velocity profiles independently. Representative goals of the optimization are to reduce cycle time and to increase overall efficiency by optimizing the respective residual times of a unit of liquid at the different temperature zones. The ability to control the temperature and velocity profiles independently using electrokinetics represents a substantial advantage of EK-PCR over PCR performed, for example, in a conventional Rayleigh-Benard convection cell (Krishnan et al., Science 298:793, 2002). The voltage dependency and spatial distribution of EK-induced fluid motion have been characterized (FIG. 4A-4B).

The respective residual times in each temperature zone can be controlled by adjusting the temperature profile using heat sinks thermally contacting the chamber. Specifically, metal electrodes (e.g., Au), which have a higher thermal conductivity than the walls or other structures in the chamber, are situated in the chamber and serve as heat sinks to increase local heat loss. Thus, the residual time in each temperature zone and the total cycle time are adjusted. The heat-sink electrodes may also serve as impedance-sensing electrodes for real-time monitoring of the product (see below for discussion of embodiments that include impedance sensing).

In addition, the width of the chamber may be adjusted to change cycle times. For example, in some embodiments, the width may be adjusted to between 2-8 mm. The volume of the chamber can be established by adjusting the channel height (within a range that will support laminar flow of liquid in the chamber). In one embodiment, the width is adjusted to between 2-8 mm while the volume of the chamber is maintained at 320 μl by making respective adjustments to chamber height. For an average velocity of 100 μm/s (based on a preliminary study), a 30-cycle EK-PCR can be completed in 15-60 minutes (each cycle is 0.5-2 minutes). The configuration (shape, location, width, and thickness) of the heating electrodes and of the heat-sink electrodes may be systematically adjusted to optimize the temperature profile and amplification efficiency. If the heat loss is small, the heat-dissipating capacity will increase with the width, thickness, and number of heat-sink electrodes. If the temperature is reduced, a higher voltage may be applied to increase the heat flux. The actual configurations will be guided by the heat equation and can be readily tested experimentally. Dual-tracer fluorescence thermometry and particle-image velocimetry can be performed to characterize the temperature and velocity profiles of the chamber.

### Fourth Representative Embodiment

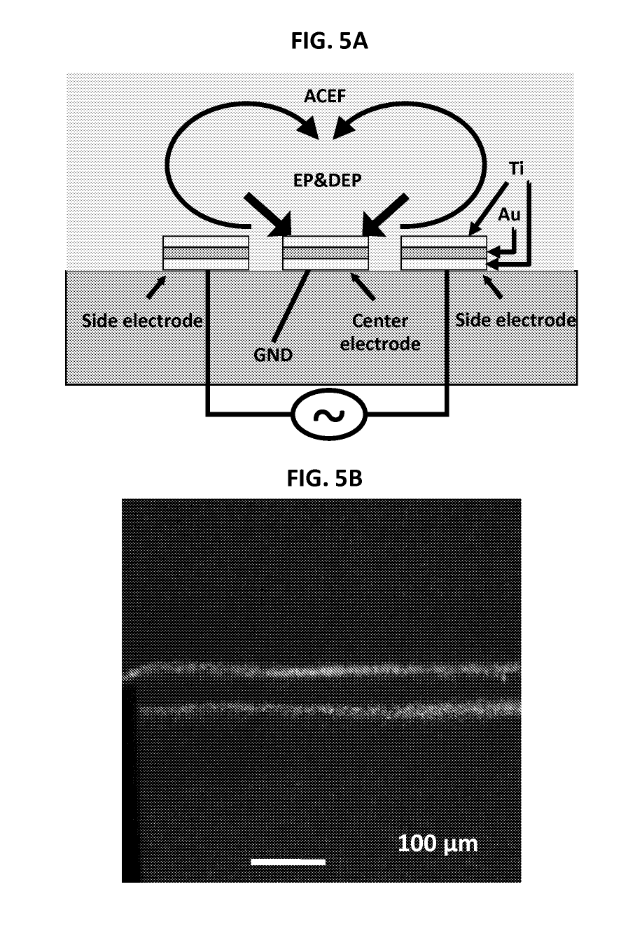

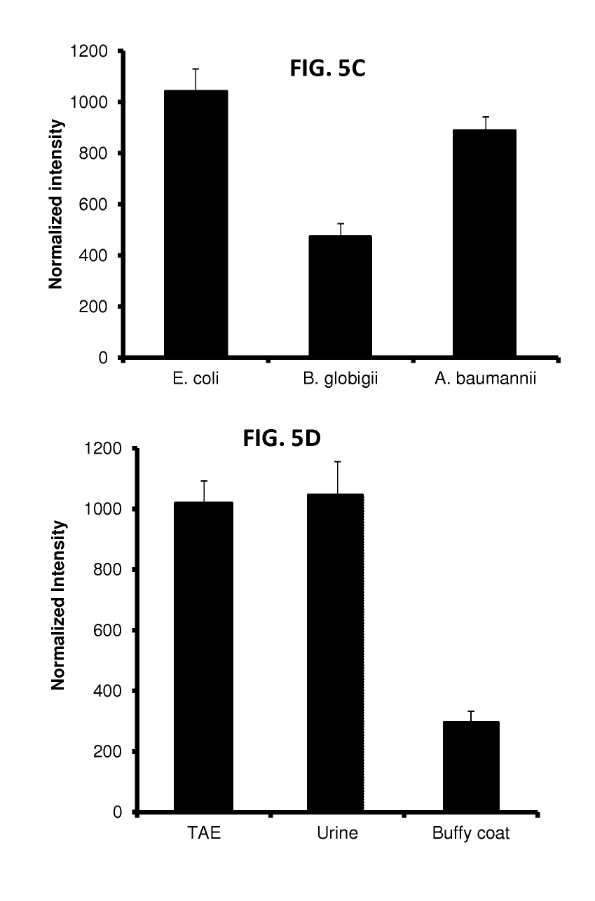

FIG. 5A depicts an electrokinetic concentrator according to this embodiment with a 3-parallel electrode configuration that combines long-range AC electrokinetic fluid motion (ACEF) and local trapping forces, including electrophoresis (EP) and dielectrophoresis (DEP). A titanium-gold-titanium sandwich was used to form stable electrodes for electrokinetic operation. As shown in FIG. 5A, the fluid motion generated by ACEF entrains and carries the target (e.g., a suspension of bacteria) towards the center electrode surface, where DEP is most effective. From there, EP and DEP bring the target further into proximity with the center electrode.

### Real-Time Impedance Sensing

In various embodiments, at least one impedance-measurement electrode may integrated into the chamber to allow real-time monitoring of the EK-PCR amplicon concentration and quantification of the initial target concentration. A heat-sink electrode may serve as an impedance-sensing electrodes for in situ monitoring of amplicon production during the EK-PCR cycle (i.e., qPCR). PCR products will hybridize to specific capture probes (e.g., complementary nucleotide sequences) which may preferably be pre-immobilized on the sensing electrodes. The fraction of amplicon captured on the electrode surface is a function of the total concentration of the product, local temperature near the electrode, and the free energy of the hybridization reaction, and may be estimated by:

$$α = \frac{1 + {C}_{T} K - \sqrt{1 + 2 {C}_{T} K}}{{C}_{T} K} ,$$

where C_{T} is the total concentration of nucleic acid strands in the solution, and K is the equilibrium constant of the hybridization reaction. The change in impedance upon DNA hybridization may be estimated by considering an equivalent circuit model (FIG. 4C). Generally, the association of probe and amplicon reduces capacitance (C_{surf}) and leak resistance (Rₗₑₐₖ) on the electrode surface. The circuit model agrees with results demonstrating the applicability of impedance sensing for in situ monitoring of amplicon production (FIG. 4D). The sensitivity of impedimetric sensing may be affected by, for example, the surface area of the electrode, the distance between two electrodes and other features of the two electrodes. Thus, these parameters may be systematically adjusted to optimize the sensing performance. In various embodiments, in situ EK noise reduction may be included to reduce matrix effects on electrochemical or impedimetric sensing.

### Assay Protocol Optimization

In various embodiments, the optimal EK-PCR conditions for detecting an EK-PCR product such as may be determined. The following is an assay protocol for determining optimal EK-PCR conditions for detecting bacterial 16S rRNA. The assay protocol may be performed using E. coli clinical isolates as a model system, and the results may be benchmarked against a standard benchtop PCR thermal cycler. RNAeasy Mini Kit (Qiagen 74104) may be used to substitute EK-based RNA isolation to evaluate the performance of EK-PCR. The current EK-PCR assay protocol for bacterial 16S rRNA is as follows:

(1) Mix 5 μL of isolated RNA with 1 μL primer solution (2 μM gene-specific primer, 10 mM dNTP mix) and 4 μL of DEPC-treated water to generate 10 μL of RNA/primer mix;

(2) Incubate the RNA/primer mix at 65° C. for 5 minutes, then chill for at least 1 minute;

(3) Add 10 μL of cDNA Synthesis Mix (2 μL of 10×RT buffer, 5 μL of 25 mM MgCl₂, 2 μL of 0.1 M DTT, 1 μL of RNaseOUT (40 U/μL) and 1 μL of SuperScript™ III RT (200 U/μL)) to the RNA/primer mixture;

(4) Incubate the synthesis/RNA/primer mixture 50 minutes at 50° C. to perform reverse transcription;

(5) Terminate the reverse transcription reaction at 85° C. for 5 minutes, then chill the resulting DNA templates;

(6) Mix 2 μL of DNA template with PCR master mixture (5 μL of 10×PCR Buffer, 1 μL of 10 mM dNTP mixture, 1.5 μL of 50 mM MgCl₂, 1 μL of 1M NaCl, 1 μL of Reverse Primer (10 μM), 0.2 μL of Platinum Taq DNA Polymerase, 37.3 μL of autoclaved distilled water) from the Invitrogen Platinum Taq kit to make 50 μL of PCR reagent mix;

(7) Deliver the PCR reagent mix into the EK-PCR chamber and activate the EK-PCR process; and

(8) Harvest the PCR amplicon for downstream detection.

### Example 1

### Concentrating of Bacterial DNA Using an EK Concentrator

This example demonstrates that hybrid EK established using EP, DEP, and ACEF can successfully be used to concentrate different types of bacteria from a variety of different biological media. This example further demonstrates that detection of bacterial DNA using EK-PCR is more time-efficient than conventional PCR, that the voltage and length of the EK-PCR chamber control the temperature gradient in the chamber, and that impedance sensing effectively quantifies the bacterial DNA amplified using EK-PCR.

FIGS. 1B-1D show results obtained using an EK concentrator embodiment with two electrodes such as that depicted in FIG. 1A. FIG. 1B is a plot of the percentage of a target bacteria captured as a function of time, showing that over 99% of the pathogens were concentrated in less than 20 minutes. FIG. 1C is is a bright-field image of blood cells, showing the blood cells urged away from the central electrodes of the concentrator shown in FIG. 1A. FIG. 1D is a fluorescence image of fluorescently stained E. coli, specifically showing E. coli concentrated between the electrodes of the concentrator shown in FIG. 1A. Remarkably, blood cells experienced negative dielectrophoresis which caused them to be pushed away from the electrode, while E. coli cells experienced positive dielectrophoresis, which caused them to be drawn into the gap between the electrodes. Therefore, the hybrid EK device is capable of simultaneous target concentration and isolation.

E. coli concentration was also performed using the 3-parallel electrode EK concentrator depicted in FIG. 5A. FIG. 5B is an electrofluorescence image showing fluorescently stained E. coli concentrated most heavily adjacent to a top surface of the center electrode. In addition, the EK concentrator was capable of concentrating other types of bacteria, including B. globigii and A. baumannii. FIG. 5C is a bar graph showing the concentration of samples of three bacteria species in the vicinity of the electrodes using the embodiment of FIG. 5A. As shown in the figure, E. coli was concentrated over 1000-fold, B. Globigii concentrated over 400-fold, and A. Baumannii concentrated around 900-fold. Also, electrokinetic separation of E. coli was achieved using different types of media, including TAE buffer, urine and buffy coat. FIG. 5D is a bar graph of an exemplary increase in E. coli concentration in the vicinity of the electrodes of over 1000-fold in TAE (Tris-acetate-EDTA) buffer, over 1000-fold in urine, and over 300-fold in a buffy coat specimen using the embodiment of FIG. 5A.

### Example 2

### Amplification of Bacterial DNA Using EK-PCR

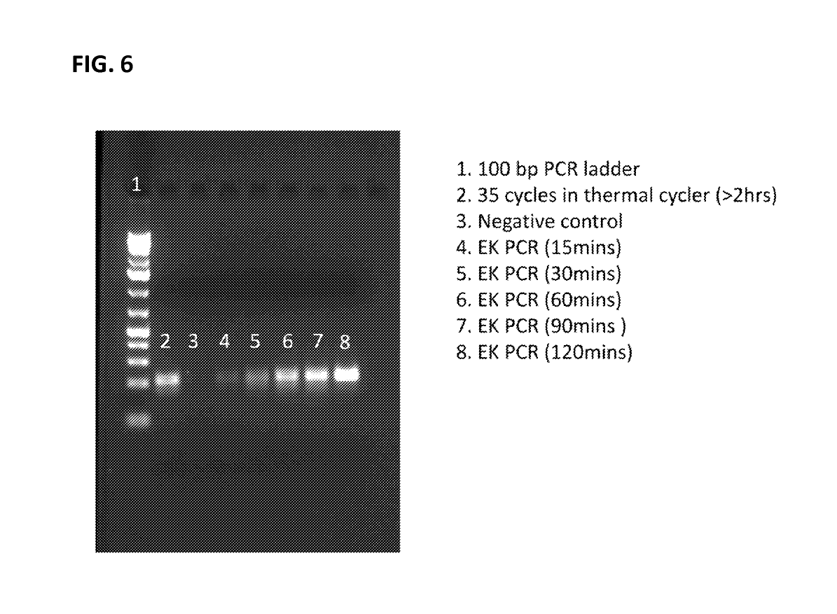

FIG. 6 shows an electrophoretic characterization of EK-PCR amplicons produced from E. coli DNA. PCR performed in a standard bench top thermal cycler for 35 cycles was compared to EK-PCR performed for 15, 30, 60, 90 or 120 minutes in a chamber as described herein. Conventional PCR required two hours to complete the 35 cycles and resulted in detectable PCR amplicon. Amplicons produced in a EK-PCR chamber were also detected for all durations tested. A similar level of amplicon production was obtained after 60 minutes of EK-PCR and after 120 minutes using a conventional thermal cycler, demonstrating that EK-PCR is more efficient than PCR performance using a standard thermal cycler.

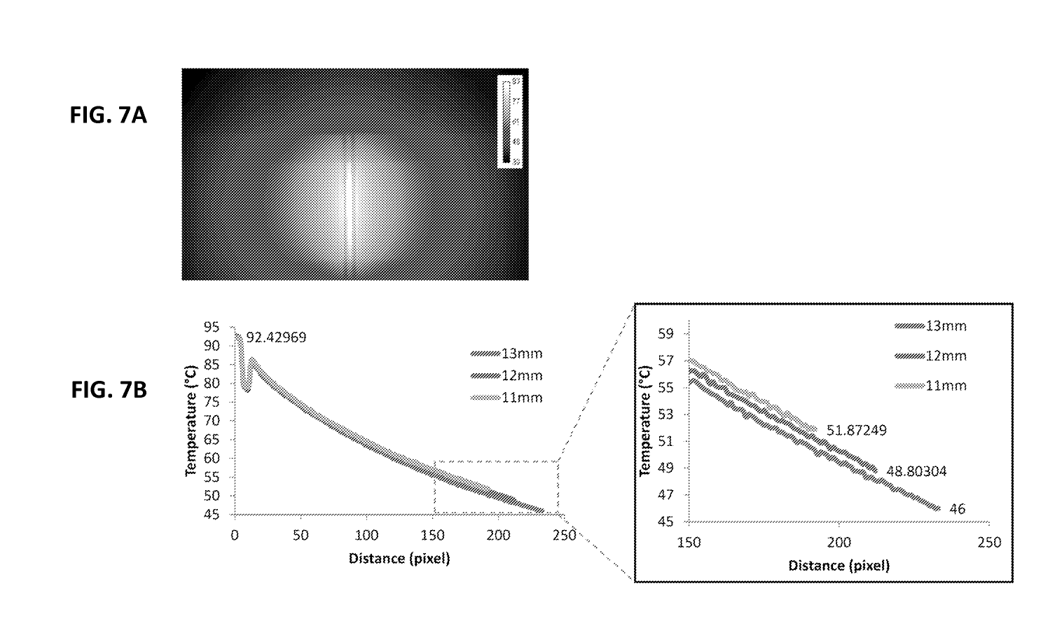

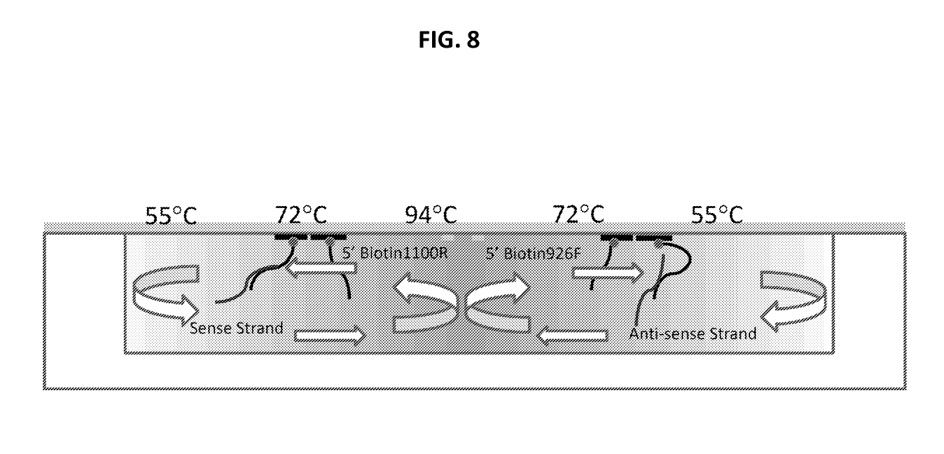

FIG. 7 demonstrates that the temperature profile in the EK-PCR chamber can be adjusted independently by changing the voltage and for length of the chamber. For example, adjusting the length of the chamber from 11 mm to 12 mm and 13 mm changed the annealing temperature from about 46° C. to about 49° C. and about 52° C., respectively (see magnified view of temperature in the annealing zone on the bottom right of FIG. 7).

### Example 3

### Real-Time Quantification of Amplification Products

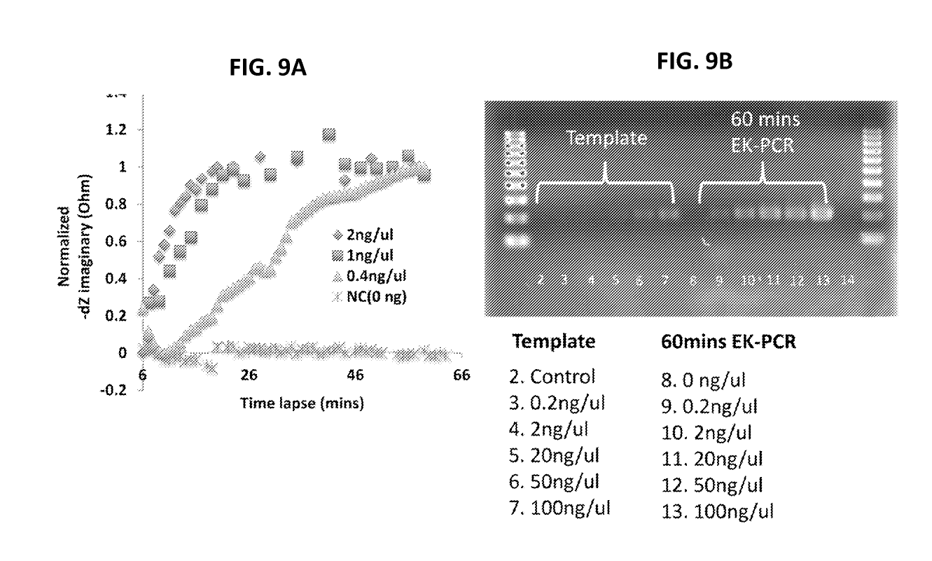

FIG. 9 shows time-lapse impedance data of real-time EK-PCR. The plot (left) shows the amount of time required for the impedance value to reach a threshold. As shown in the plot, the time required to reach threshold increased with progressively higher initial template concentrations (control, 0.4 ng/μL, 1 ng/μL and 2 ng/μL). This provides means for quantifying the template DNA concentration by, for example, fitting the impedance data to plots known to correspond to respective initial template concentrations. A gel electrophoresis photograph (right) shows correct amplification of the amplicon following 60 minutes of EK-PCR. Amplified amounts were demonstrated in proportion to initial template concentrations (control, 0.2 ng/μL, 2 ng/μL, 20 ng/μL, 50 ng/μL and 100 ng/μL). In other words, the brightest bands in the initial template electrophoresis remained the brightest bands after EK-PCR.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An enclosure for performing polymerase chain reaction (PCR), comprising:
walls defining an enclosed chamber in which an electrically conductive liquid can be contained that is formulated for performing PCR; and
a first set of electrodes contacting the conductive liquid in the chamber, the first set of electrodes when electrically energized causing localized heating of the conductive liquid between the electrodes of the first set to produce an electrokinetic convective circulation of the liquid in the chamber, the convective circulation defining at least three temperature zones in the liquid in which denaturation, annealing, and extension phases, respectively, of PCR occur on a sample of genetic material being carried by the circulating liquid successively to the temperature zones.

2. The enclosure of claim 1, wherein the denaturation temperature zone extends at least between the electrodes.

3. The enclosure of claim 1 or claim 2, wherein:
the first set of electrodes flanks a cross-dimension of the base to define first and second bilateral regions in the chamber; and
the energized electrodes cause the liquid in the chamber to circulate in the first and second regions, respectively, in a bilaterally symmetrical manner to produce first and second, respectively, electrokinetic convective circulations of the liquid in the chamber.

4. The enclosure of claim 3, wherein:
the convective circulation of the liquid in the chamber exposes the liquid to repeated cycles of the annealing, extension, and denaturation temperatures.

5. The enclosure of claim 1, wherein the chamber is dimensioned to support laminar flow of the liquid in the chamber.

6. The enclosure of claim 1, wherein the denaturation temperature zone has the highest temperature, the annealing temperature zone has the lowest temperature, and the extension temperature zone has an intermediate temperature.

7. The enclosure of claim 1, wherein the denaturation zone has a temperature between 94° C. and 98° C., the extension zone has a temperature of between 72° C. and 80° C., and the annealing zone has a temperature of between 50° C. and 65° C.

8. The enclosure of claim 1, wherein the denaturation temperature zone is about 94° C., the extension temperature zone is about 72° C., and the annealing temperature zone is about 55° C.

9. The enclosure of claim 1, wherein the electrically conductive liquid comprises a PCR buffer, and the sample comprises template DNA, primers, dNTPs, and DNA polymerase.

10. The enclosure of claim 1, further comprising at least one amplicon-sensing electrode having respective surface(s) contacting the liquid inside the chamber, the surface(s) comprising molecules of a nucleic-acid probe immobilized thereon, the probe being reactive to amplicons produced by the PCR occurring in the chamber, and the amplicon-sensing electrode exhibiting an impedance change with a corresponding change in amount of amplicon bound to the electrode(s).

11. The enclosure of claim 1, further comprising a second set of electrodes contacting the conductive liquid in the chamber, the second set of electrodes when electrically energized producing a hybrid EK flow profile urging movement of particles of a biological sample toward the second set of electrodes, thereby locally concentrating the sample.

12. The enclosure of claim 11, further comprising an elution electrode contacting the conductive liquid in the chamber, the elution electrode when electrically energized urging elution of the particles from the chamber.

13. The enclosure of claim 11, wherein the second set of electrodes comprises a grounded electrode flanked by electrodes of the second set that are connected to an AC source.

14. The enclosure of claim 11, further comprising a third set of electrodes including respective surfaces contacting the liquid inside the chamber, the third set of electrodes when electrically energized producing a hybrid EK flow profile urging movement of particles of a biological sample toward the third set of electrodes, thereby locally concentrating the sample in the chamber.

15. The enclosure of claim 1, further comprising at least one heat-sink electrode in thermal contact with the liquid in the chamber, the heat-sink electrode imposing a corresponding change to the electrokinetic convectional circulation.

16. An electrokinetic chamber, comprising:
a base, sides, and a cover defining an enclosed chamber in which an electrically conductive biological liquid can be contained, the chamber being dimensioned to support laminar flow of the biological liquid in the chamber;
a first set of electrodes situated inside the chamber and flanking a cross-dimension of the base to define first and second bilateral regions in the chamber, the first set of electrodes when electrically energized producing a dielectrophoresis force profile extending from the vicinity of the electrodes and urging movement of particles in the liquid toward the electrodes of the first set, thereby locally concentrating the particles in the liquid in the vicinity of the electrodes.

17. The chamber of claim 16, wherein energization of the first set of electrodes further produces a flow of the liquid that, in cooperation with the dielectrophoresis force profile, locally concentrates the particles.

18. The chamber of claim 16, wherein the particles comprise biological cells.

19. The chamber of claim 16, further comprising a second set of electrodes contacting the liquid in the chamber, the second set of electrodes when electrically energized causing localized heating of the conductive liquid between the electrodes of the first set to produce an electrokinetic convective circulation of liquid in the chamber, the convective circulation defining at least three temperature zones in the liquid in which denaturation, annealing, and extension phases, respectively, of PCR occur on a sample of genetic material being carried by the liquid successively to the temperature zones.

20. The chamber of claim 19, further comprising at least one amplicon-sensing electrode having a surface contacting the liquid inside the chamber, the surface comprising molecules of a nucleic-acid probe immobilized thereon, the probe being reactive to amplicons produced by the PCR occurring in the chamber, and the amplicon-sensing electrode exhibiting an impedance change with a corresponding change in amount of amplicon bound to the electrode.

21. The chamber of claim 16, further comprising at least one heat-sink electrode in thermal contact with the liquid in the chamber, the heat-sink electrode imposing a corresponding change to the electrokinetic convectional circulation.

22. A system, comprising:
a first enclosed chamber hydraulically connect to a second enclosed chamber such that liquid eluted from the first chamber enters the second chamber,
the first chamber comprising a first set of electrodes situated inside the first chamber so as to contact a liquid in the first chamber, the first set of electrodes when electrically energized producing a hybrid electrokinetic force profile in the liquid urging movement of particles in the liquid toward the first set of electrodes, thereby concentrating the particles in said vicinity; and
the second chamber comprising a second set of electrodes situated inside the second chamber so as to contact a liquid in the second chamber, the second set when electrically energized producing localized heating of liquid between the electrodes and a resulting electrokinetic convection at circulation of the liquid in the second chamber, the electrokinetic circulation defining at least three temperature zones in second chamber in which denaturation, annealing, and extension phases, respectively, of PCR are performed on a sample of genetic material suspended in the liquid and being carried out by a liquid successively to the temperature zones.

23. The system of claim 22, further comprising a third set of electrodes, situated in the second chamber in contact with the liquid, the third set comprising a nucleic-acid probe to which amplicons produced by the PCR are attracted and bound so as to alter an impedance across the third set of electrodes as a function of amount of amplicon bound to the electrodes.

24. The system of claim 23, wherein the third set of electrodes has a heat-sink property sufficient to alter a respective position of at least one of the three temperature zones.

25. A diagnostic method, comprising:
introducing a nucleic-acid containing sample, including a template DNA, a selected primer, dNTPs, and a nucleic acid polymerase, to an electrokinetic (EK)-convection circulation of liquid defining the PCR denaturation-temperature zone, the PCR annealing-temperature zone, and the PCR extension-temperature zone in the electrokinetic chamber of claim 19; and
allowing the convection circulation of liquid to transport the sample successively to the denaturation-, annealing-, and extension-temperature zones in multiple cycles to produce amplicons of the template nucleic acid.

26. The method of claim 25, further comprising, before introducing the sample to the EK-convection circulation, concentrating the sample by subjecting the sample to a hybrid-EK force profile.

27. The method of claim 25, further comprising causing localized dissipation of heat from the chamber, thereby altering a respective position of at least one of the temperature zones.

27. The method of claim 25, further comprising monitoring amplicon production in the EK-convective cycle by:
binding amplicons to surfaces of electrodes comprising molecules to which amplicons bind; and
monitoring an electrical impedance of the electrodes as a function of amount of bound amplicons.

28. The method of claim 25, further comprising identifying at least one produced amplicon.

29. The method of claim 28, wherein the at least one amplicon is identified by gel electrophoresis or a DNA probe specific for the amplicons.
